(19) United States
(12) Patent Application Publication
(10) Pub. No.: US 2023/0389872 A1
(43) Pub. Date: Dec. 7, 2023

(54) FOOT MOUNTED SYSTEM FOR OPERATING A MEDICAL DEVICE

(71) Applicant: Jason Salsamendi, Trabuco Canyon, CA (US)

(72) Inventor: Jason Salsamendi, Trabuco Canyon, CA (US)

(21) Appl. No.: 17/805,391

(22) Filed: Jun. 3, 2022

(51) Int. Cl. A61B 5/00

(52) U.S. Cl. CPC A61B 5/6807; A61B 2560/0487; A61B 2562/0247; A61B 2017/00411

(57) ABSTRACT

A system for controlling one or more functions of medical equipment comprises a plurality of sensors and/or switches carried on one or both shoes of a clinician and operable without requiring interaction by hands of the clinician. Through a primary input, the clinician can actuate a first sensor in order to select one of the functions of the medical equipment. A secondary input from the clinician, in which a sensor provides an actuation signal, will actuate the selected medical function.

### CROSS-REFERENCE TO RELATED APPLICATIONS

Not Applicable.

### STATEMENT RE: FEDERALLY SPONSORED RESEARCH/DEVELOPMENT

Not Applicable.

### BACKGROUND

The various aspects and embodiments described herein relate to control interfaces for medical devices.

Many surgical procedures are performed using multiple instruments. For example, in a catheterization laboratory, a clinician advances a catheter through a patient's vasculature guided by fluoroscopy, which entails turning radiation-emitting x-ray equipment on and off. Foot pedals can be used to enable the clinician to control the x-ray equipment without using his or her hands, which are being used for managing the catheter. Other medical devices, such as electrocautery devices, robotic arm systems, and laser cutters, can be at least partially controlled by a foot pedal interface, and thus in some instances multiple medical devices may be controlled by foot pedals during a single procedure.

While foot pedals are serviceable, they can be cumbersome to use. Their placement may be inconvenient, requiring that they be moved or that the clinician move or contort to operate the foot pedal. Further, there is a risk of medical staff unintentionally contacting foot pedals, causing the medical staff to stumble and/or undesirably actuate medical devices. Other objects, such as operating room tables, may also unintentionally contact foot pedals, leading to undesirable actuation of medical devices and potential damage to medical equipment, clinicians, and/or patients.

### BRIEF SUMMARY

The various aspects described herein relate to a foot-mounted system for operating a medical device. This system can be integrated into a pair of shoes to be worn by a clinician, such as the physician performing the procedure. The pair of shoes may include a left shoe and a right shoe. At least one of the left and right shoes may have one or more switches. The switch on the left or right shoe may be activated with the other foot. Activating the switch selects, but does not necessarily actuate, a function of the medical device. The selected function of the medical device may be activated upon sensing a threshold pressure at one or more pressure sensors at a bottom side in the left and/or right shoe.

By way of example and not limitation, the left shoe may have a first switch. The first switch is operative to select function one of the medical device. A clinician may activate the first switch by moving his or her right foot close to the first switch while standing on the left foot. The first switch may be a proximity sensor, button, or the like. When the first switch senses the clinician's right foot in close proximity to the first switch and the weight of the clinician is sensed by a sensor in the left shoe, the first switch is activated, selecting function one of the medical device. To actuate, or turn on, selected function 1 of the medical device, the clinician may shift his or her weight to both feet, then lean forward to apply increased pressure to the ball of the clinician's feet. In a normal stance, the heels and balls of the clinician's feet feel pressure. The pressure sensors in the left and right shoes are located at the ball of the feet. When the clinician stands normal, typically, his or her weight will be distributed on the heels of the feet and on the balls of the feet. Thus, the pressure sensors in the left and right shoes will each sense less than a threshold percentage (e.g, 20% for each of the pressure sensors) of the clinician's weight.

When the clinician shifts his or her weight to the front of their feet, the pressure sensors each will sense a weight greater than the threshold percentage (e.g., 20%) of the clinician's weight. When both of the pressure sensors in the left and right shoes sense a weight above the threshold percentage of the clinician's weight, selected function 1 of the medical device may be activated.

When one or both of the sensors in the left and right shoes senses a weight below the threshold percentage of the clinician's weight, selected function 1 of the medical device may be deactivated. Function 1 of the medical device may not be activated if only one of the sensors in the left and right shoes sense a weight above the threshold percentage of the clinician's weight.

The threshold percentage of the clinician's weight is described as being 20% of the clinician's weight. However, it is also contemplated that other threshold percentages are also contemplated such as between 10% and 90% and any increment of 1% therebetween.

The left and/or right shoes may have a wireless transmitter/transceiver that is in communication with the medical device. Such communication may be operative to activate and deactivate the function of the medical device.

The left and/or right shoes may have a feedback structure such as a vibration device for providing haptic feedback. The vibration device vibrates when any function of the medical device activatable by the left and/or right shoes is selected. This is to provide the clinician with a sensory feedback to indicate that a function of the medical device has been selected.

The various aspects have been described in relation to a medical device. However, it is also contemplated that a function of any machine, computer, software, or mobile software application may be operated as described herein with the shoes. It is also contemplated that an attachment with similar structure can be attached to the user's own shoes to provide similar function in lieu of the system being embedded into the shoes of the clinician.

In accordance with one embodiment, the present specification provides a medical system comprising a medical equipment having a function. The medical equipment is configured so that the function can be changed from a first operational state to a second operational state. A system interface is in communication with the medical equipment. A switching mechanism is configured to be carried on one or more of a user's shoes. The switching mechanism comprises a first input structure, a processor, and a transceiver. The first input structure is configured to be actuated by being put in contact or close proximity with a second user shoe, and is configured to communicate a first function actuation signal to the processor when actuated. A trigger input structure is actuable by the user further manipulating movement of the first user shoe, the trigger input structure configured to communicate trigger data to the processor. The processor is configured to select a first function upon receiving the first function actuation signal, and is further configured to generate a first control instruction when the trigger data satisfies a trigger criteria and the first function is selected. The transceiver is configured to wirelessly transmit the first control instruction to the system interface. The medical equipment is configured to transition the first function from the first operational state to the second operational state in response to the system interface receiving the first control instruction.

In some versions the switching mechanism is incorporated into one of the user's shoes.

In other versions the switching mechanism is releasably attachable to at least one of the user's shoes.

In additional versions the switching mechanism additionally comprises a second input structure configured to be actuated by being put in contact or close proximity with the second user shoe and configured to communicate a second function actuation signal to the processor when actuated.

In some such versions the processor is configured to select a second function upon receiving the second function actuation signal, and the processor is configured to generate a second control instruction when the trigger data satisfies the trigger criteria and the second function is selected.

In further versions, the switching mechanism is incorporated into a first shoe of a of the user's pair of shoes, and a second switching mechanism is incorporated into a second shoe of the user's pair of shoes, the second switching mechanism comprising a third input structure, a second processor, and a second transceiver, the second processor configured to communicate with the processor of the switching mechanism.

In yet further versions, the switching mechanism additionally comprises a movement sensor in communication with the processor, and wherein the processor is configured to determine whether the user is moving away from the medical equipment based on data from the movement sensor.

In still further versions, the switching mechanism additionally comprises a feedback structure configured to provide notice to the user when a function of the medical equipment has been selected.

In yet additional versions the processor is configured to de-select the first function upon receiving the first function actuation signal when the first function is already selected.

A still further version can additionally comprise an electronic interface configured to communicate with the medical equipment and communicate wirelessly with the transceiver of the switching mechanism.

In accordance with another embodiment, the present specification provides a method for hands-free control of a medical equipment, comprising receiving a primary input from a clinician, the primary input comprising the clinician actuating a first input structure carried on a first shoe of the clinician to select a first function of the medical equipment. A feedback can be generated to notify the clinician that the first function has been selected. A secondary input is received from the clinician, the secondary input comprising the clinician actuating a trigger input structure carried on the first shoe of the clinician. The selected first function of the medical equipment is placed in an operational state upon receiving the secondary input, and is maintained in the operational state for as long as the trigger input structure is actuated.

In an additional version, the trigger input structure comprises a pressure sensor.

A further version comprises the clinician actuating the trigger input structure by shifting weight forwardly.

Some such versions comprise the processor determining that the trigger input structure is actuated when a pressure data from the pressure sensor satisfies an actuation criteria.

Further such versions can additionally comprise receiving a second primary input from the clinician, the second primary input comprising the clinician actuating a second input structure carried on a second shoe of the clinician, and in response to the second primary input de-selecting the first function and selecting a second function of the medical equipment.

In some versions, receiving the secondary input comprises the clinician actuating the trigger input structure carried on the first shoe and actuating a second trigger input structure carried on the second shoe of the clinician. The second trigger input structure can comprise a second pressure sensor, and a second processor carried on the second shoe of the clinician can determine that the second trigger input structure is actuated when a second pressure data from the second pressure sensor satisfies the actuation criteria.

Further versions can additionally comprise the second processor electronically communicating that the second pressure sensor satisfies the actuation criteria to the processor of the first shoe.

Yet further versions comprise placing the second function of the medical equipment in an operational state when the pressure sensor of the first shoe satisfies the actuation criteria and the second pressure sensor also satisfies the actuation criteria.

In still additional versions, generating the feedback comprises activating a haptic feedback that can be felt by a foot of the clinician wearing the first shoe.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic side view of a left shoe configured in accordance with an embodiment;

FIG. 2 is a schematic side view of a right shoe configured in accordance with the embodiment and useable with the left shoe of FIG. 1;

FIG. 3 is a schematic top view showing electronic components incorporated into the shoes of FIGS. 1 and 2;

FIG. 4 is a box diagram showing the interaction of components in an embodiment;

FIG. 5 is a schematic top view showing the shoes of FIGS. 1 and 2 during operation;

FIG. 6 is a flow chart showing operation of the system in accordance with an embodiment;

FIG. 7 is a perspective view of an embodiment of a device configured to be releasably attached to a left foot and/or left shoe of a user; and

FIG. 8 is a side view of the device of FIG. 6 attached to a shoe (which is shown in phantom lines).

### DETAILED DESCRIPTION

This specification describes embodiments and versions of a foot-mounted system for operating one or more functions of a medical equipment during a medical procedure. As will be discussed in more detail below, this system can be integrated into or carried by a pair of shoes to be worn by a clinician, such as the physician performing the procedure. At least one of left and right shoes may have one or more switches that, when actuated, provides a primary input that selects but does not necessarily actuate a function of the medical equipment. The selected function of the medical equipment may be activated upon receiving a secondary input. Such a secondary input may be, for example, sensors detecting that the clinician has shifted his or her weight forwardly.

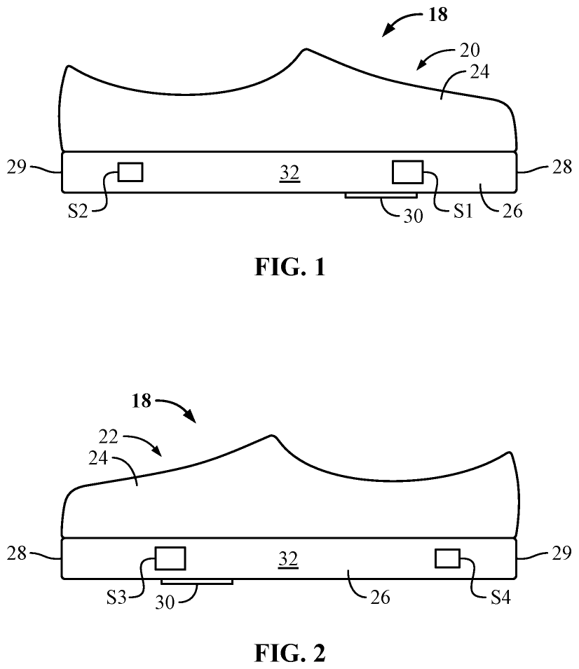

With initial reference to FIGS. 1 and 2, a system 18 for controlling medical equipment 60 comprises a left shoe 20 and a right shoe 22 that are configured to be worn by a clinician during a medical procedure. Each of the shoes 20, 22 has an upper 24 that extends upwardly from a sole 26, and each shoe extends from a toe 28 to a heel 29. A trigger sensor 30, which preferably comprises a pressure sensor, is formed on each sole 26 and preferably is positioned so as to be aligned with the ball of a wearer's foot. An inner side 32 of the left shoe 20 can include a first switch S1 and a second switch S2. The first switch S1 can be disposed near the toe 28, while the second switch S2 can be disposed near the heel 29. The inner side 32 of the right shoe 22 can include a third switch S3 and a fourth switch S4. The third switch S3 can be disposed near the toe 28, while the fourth switch S4 can be disposed near the heel 29.

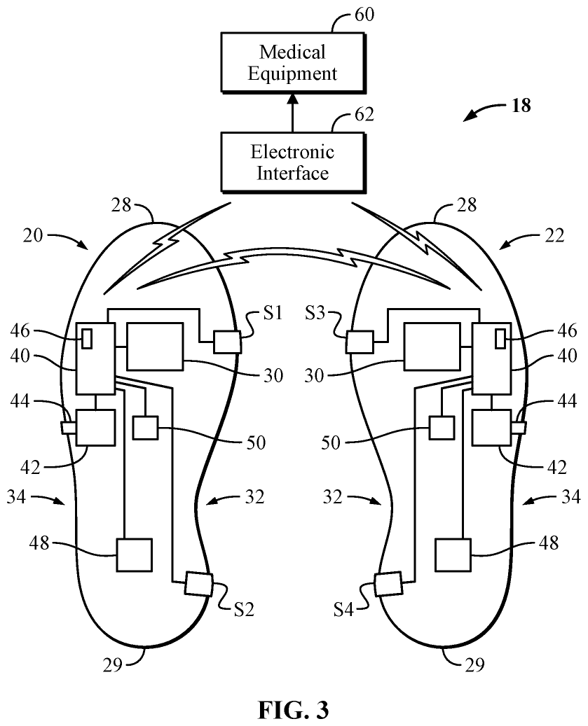

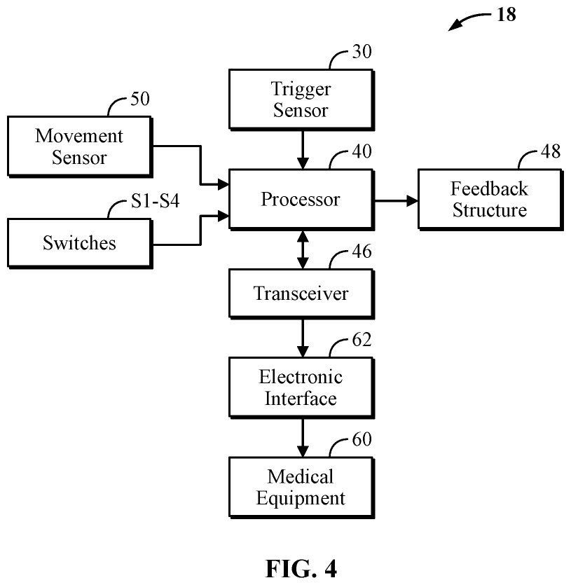

With additional reference to FIGS. 3 and 4, various electronic components can be disposed within the shoes 20, 22, and most preferably are disposed within the sole 26 of each shoe 20, 22. These components are configured to obtain and provide data to assist the wearer in controlling functions of medical equipment 60. In the illustrated embodiment, a processor 40 can be configured to electronically communicate with the first and second switches S1, S2 in the left shoe 20, while a processor 40 of the right shoe 22 is configured to electronically communicate with the third and fourth switches S3, S4. A battery 42 can be configured to supply power to the corresponding processor 40 and other components, and can communicate with a charge port 44 that can be accessible from outside the shoe, for example opening from an outer side 34. In some embodiments, rather than a physical charge port 44, the battery 42 can be charged using other technologies, such as inductive charging, and/or the battery can be physically removable and replaceable.

The trigger sensor 30 is also configured to communicate with the processor 40. In a preferred embodiment the trigger sensor 30 comprises a pressure sensor. The trigger 30 provides a signal to the processor 40 indicating that pressure is applied and providing a measurement of the amount of pressure applied thereto. As will be discussed in more detail below, the processor 40 can use this information to prescribe instructions.

A transceiver 46 is in communication with the processor 40 and preferably is configured to send and receive wireless data. The transceiver 46 can be configured to operate using any of a variety of technologies, such as Wi-Fi, Bluetooth, near field communication (NFC), or the like. In this manner, the processors 40 of each shoe can wirelessly communicate with one another and with other components, including third-party components.

A feedback structure 48 in electronic communication with the processor 40 can be configured to provide sensory feedback to the wearer of the shoe to notify the wearer of a condition or action. In a preferred embodiment, the feedback structure 48 comprises a haptic actuator such as a vibrating structure configured to provide a tactile effect that will be felt by the wearer. In additional variations, the feedback structure 48 can provide other forms of notification, such as an aural effect (i.e., sound from speaker) and/or a visual effect (e.g., light emitting diode light). The vibrating structure can provide feedback to the user by vibrating once, twice, thrice, etc., vibrating continuously, vibrating increasingly, vibrating intermittently, or the like. Different feedback patterns can be provided for different situations. For example, selection of a function can include a single, one-second vibration feedback. However, when the medical equipment function is activated, the vibration device may provide a continued vibration or a series of short, spaced-apart vibration bursts that continues the entire time the function is activated. De-selection of a function may include, for example, a pair of short vibration bursts. In some versions, each function may have its own unique vibration pattern when selected. For example, the first function may have a single, short vibration; the second function may have two short, spaced-apart vibrations, and so on to the fourth function, which may have four short, spaced-apart vibrations to indicate when it is selected.

An optional movement sensor 50, also in communication with the processor 40, is configured to detect motion of the corresponding shoe 20 that would suggest, for example, that the wearer no longer intends to use the shoes to control medical equipment. For example, in one embodiment, the movement sensor 50 is an accelerometer or gyroscope. When the processor 40 receives data from the movement sensor 50 that indicates that, for example, the wearer of the shoes 20, 22, has moved away from certain medical equipment 60 and/or is walking for a substantial distance, the processor 40 can be configured to deactivate features of the shoes 20, 22. Similarly, during periods in which no motion is detected by the movement sensor 50, the processor 40 can determine that the shoes 20, 22 are not being worn, and direct the system 18 to be in a “sleep” mode to save energy. However, if a clinician puts on the shoes 20, 22, the signals from the movement sensor 50 may prompt the processor 40 to wake up the system 18.

With continued reference to FIGS. 3 and 4, the illustrated transceiver 46 can be configured to wirelessly communicate with an electronic interface 62 that is further in electronic communication with medical equipment 60. Preferably, the medical equipment 60 includes one or more functions. It is to be understood that the medical equipment 60 may in fact include multiple different medical devices, each having one or more different functions that are desirably controlled by the system 18. In some embodiments, the electronic interface 62 can be directly wired to the corresponding medical equipment 60. In other embodiments, a wireless data link can be employed. The electronic interface 62 can be part of the medical equipment 60 in some versions. In other versions, the electronic interface 62 can be provided in a kit with the shoes 20, 22 and can be attached and/or programmed to communicate with the medical equipment 60 through wired or wireless structures. In still further versions, the electronic interface can be integrated into one of the shoes 20, 22, making that shoe in charge of communication with the medical equipment 60. In yet further versions the electronic interface can include a computing device, such as a laptop, tablet or smartphone that is configured to communicate wirelessly with one or more of the shoes 20, 22 and with the medical equipment 60.

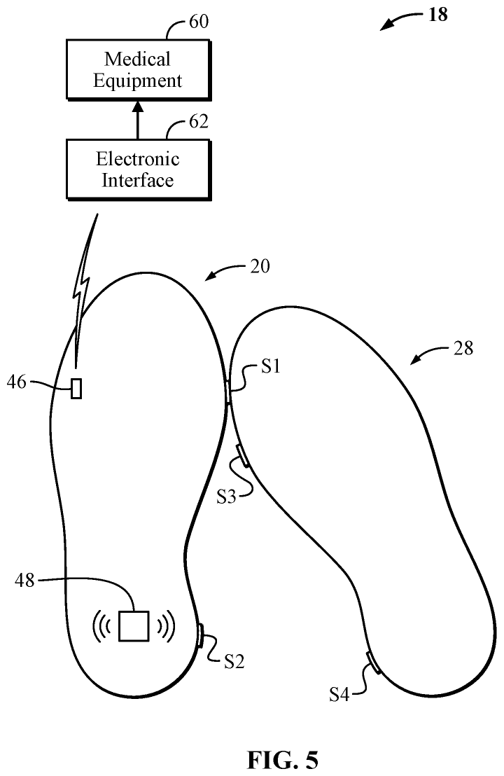

Each of the Switches S1-S4 can be actuated without the use of a wearer's hands. In some embodiments, each switch S1-S4 comprises a button switch that is actuated by physical contact with another structure depressing the button switch while the trigger sensor of that shoe is sensing the person's weight. In other versions, each switch S1-S4 can comprise a pressure sensor or a proximity sensor. As such, each switch can be actuated by bringing it into contact with or in close proximity to another object such as the opposing shoe. Of course, it is to be understood that other specific types of switches can suitably be used. FIG. 5 depicts an instance in which the first switch S1 is being actuated by the wearer bringing the right shoe 22 into contact with the left shoe 20 at the location of the first switch S1. In this situation, the trigger sensor of the left shoe senses the weight of the clinician. When the first switch S1 has been successfully actuated, the processor 40 can trigger the feedback structure 48 to signal the wearer that efforts to actuate the first switch S1 have been successful. In a preferred version, the feedback structure 48 is configured to use a first signal pattern, such as a single, one-second-long vibration, when a switch S1-S4 has been actuated.

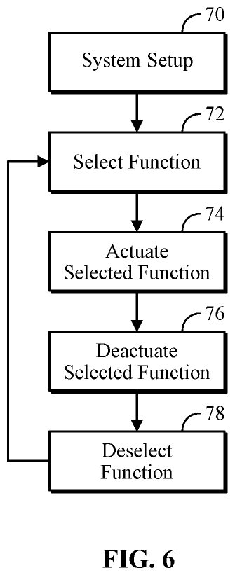

With additional reference next to FIG. 6, in operation, the system, which includes the left shoe 20 and right shoe 22, is first set up 70. System setup 70 can include establishing electronic communication between the shoes 20, 22 and the medical equipment 60, such as via the electronic interface 62. The medical equipment functions to be controlled are identified, and each such function is assigned to one of the switches S1-S4. This can be accomplished in any of several ways. For example, in some versions the processors 40 and/or electronic interface 62 can be programmed to correlate specific switches S1-S4 to specific medical equipment functions. In some embodiments the electronic interface 62 can be a computing device such as a laptop, tablet or smartphone having an app that facilitates setup 70. For example, the app can be configured to establish communication between the electronic interface 62 and both the computer equipment 60 and the shoes 20, 22. Thus, certain functions of the computer equipment 60 can be assigned to certain ones of the switches S1-S4 via the app during setup. In additional versions, the electronic interface 62 can have specific outlets that correspond to specific switches, and connecting a medical device function to a particular outlet correlates the function to the corresponding switch.

During setup 70, the clinician will activate the system 18, such as by putting on the shoes 20, 22, which, as discussed above, can trigger the shoes 20, 22 to be placed in an awake state when the trigger sensors of the shoes senses a weight greater than 2 times a weight of the shoes. In additional versions, the shoes 20, 22 may include on/off buttons. Once awake and being worn by the clinicians, the shoes 20, 22, preferably will make calibrations if needed. For example, in some versions, the shoes 20, 24 will take measurements from the trigger pressure sensors 30 and the processors 40 will determine, and save, the weight of the clinician. Such calibration can be accomplished in various ways. In some versions the wearer can enter their weight into a memory of the processor 40. In another version, wearer weight can be entered into an app on a computing device, which may communicate this data to the processor 40. In a further version the app can instruct the wearer to make various movements, such as standing on only the left shoe 20, then the right shoe 20, then on both feet in an at rest position, then leaning forward. From such measurements the app can determine the wearer's weight and/or calculate desired threshold pressures for operating the trigger sensors 30.

After system setup 70, and during the medical procedure, the clinician will next select a function 72 of the medical equipment 60. This can be accomplished by actuating one of the switches S1-S4, such as discussed herein and in connection with FIG. 5. For example, actuating the first switch S1 will select the first function, which corresponds to the first switch S1. By way of example and not limitation, actuating the first switch may be accomplished by moving the right shoe near switch S1 in the left shoe. When the right shoe is moved near switch S1, the trigger sensor located in the left shoe must also sense a weight of the clinician. In this way, the clinician can't walk (i.e., raise the left shoe) and accidentally strike an object so as to accidentally actuate switch S1. The shoe in which the switch to be activated must be stationary. The switch S1 can be a proximity sensor so that the right foot or shoe only need be placed near the switch S1. Alternatively, switch S1 may be a pressure switch so that the right shoe has to depress the pressure switch S1. In a preferred embodiment, selecting the first function does not actuate, or turn on, the first function of the associated medical equipment 60. Rather, the processor 40 actuates the feedback structure 48 so the clinician knows that the attempt to has been successful, and that the first function has been selected. It is to be understood that, in additional versions, the feedback structure 48 can take various forms. For example, in another version an individual feedback structure can be located adjacent each of the switches S1-S4. Thus, when a particular switch is actuated, the feedback structure (such as haptic feedback) at or adjacent the switch is actuated. This not only notifies the clinician that a function has been selected, but clarifies which function has been selected based on the location of the feedback. In yet additional versions, a visual indicator, such as a screen, can be supported at or adjacent the medical equipment 60 and the processor 40 can direct the screen to display an identification of the selected function and its status as selected or not selected. In a still further version, the screen can display a list of each of the functions controlled by the system along with each function's current status, such as selected, not selected, currently in operation, or the like.

Once a function of the medical equipment 60 has been selected, it can be actuated 74 by a secondary input from the clinician received through the shoes 20, 22. In a preferred embodiment, the secondary input is received through the trigger sensors 30. More specifically, the clinician can actuate the selected function by shifting his or her weight in a particular manner. In one version, when the clinician is standing normally with both feet on the floor, their weight will be generally evenly distributed across both shoes. In each shoe 20, 22, the processor 40 can compare the pressure measurement received from the associated trigger sensor 30 to the wearer weight that is stored in memory to determine the percentage of the wearer's weight supported at the location of each of the trigger sensors 30, which preferably are located aligned with the ball of the wearer's foot. If the clinician is standing normally, a majority of weight will be supported on the heels, and each trigger 30 will measure a pressure corresponding to less than a threshold pressure of, for example, 20% of the wearer's weight.

In one embodiment, the system is configured so that the selected function is actuated when the clinician, while standing on both feet, shifts his or her weight forwardly. With such a weight shift, each trigger 30 will measure a pressure greater than a threshold pressure (such as about 20% of the wearer's weight) and communicate that measurement to the corresponding processor 40. Upon recognizing that the trigger 30 measurement exceeds the threshold, each processor 40 will communicate an instruction to actuate the selected function. In some versions, both processors 40 must communicate actuation instructions to the electronic interface 62 in order for the function of the medical equipment 60 to be actuated. That is to say, the electronic interface 62 can be configured to direct actuation of the selected function only when it receives an actuating instruction from both processors 40. In other versions, the processors 40 of the left and right shoes 20, 22 will communicate with each other, and one of the processors 40 can take the lead in transmitting actuation instructions. In one example the left shoe processor can be designated a master, or primary, processor. The right shoe processor can transmit a signal to the left shoe processor indicating that the right shoe trigger 30 has measured over the threshold. If the left shoe processor also determines that the left shoe trigger has measured over the threshold, the left shoe processor can then transmit an activation signal to the interface 62 directing activation of the selected function. Preferably, the selected function remains actuated until one or both of the triggers 30 no longer measures over the threshold.

As long as the triggers 30 both measure over the threshold, the selected function remains operating. To deactivate 76, or stop operation of, the selected function, one or both of the triggers 30 will measure below the threshold. For example the clinician can simply lift one of the shoes so that the sensed weight on the lifted shoe falls below the threshold value. In such a case, the associated processor 40 will identify that the actuation condition is no longer valid, and will cease to transmit a signal to actuate the selected function. Another way the clinician can deactivate the selected function is to simply return his or her weight distribution to normal, which will also cause the trigger sensors 30 to measure a pressure below the threshold.

Notably, in some versions, during setup 72 tests may be performed to set the threshold pressure. For example, as part of calibrating the shoes 20, 22, a software application on a wearer's smartphone, tablet, laptop or other computing device can direct the wearer to assume certain positions, and the application and/or processors will note the pressure measurements at each position. For example, the application will direct the clinician to stand normal in an at-rest position, and will note the weight measurements of the trigger sensors 30. The app will further direct the clinician to lean forward so as to shift weight forward to a position desired to actuate a selected function, and will again note the weight measurements of the trigger sensors 30. Further positions, such as standing on one foot or the other, can also be calibrated. The application or the processors 40 can take these calibration measurements and calculate a threshold pressure for each shoe 20, 22 that will be used by the associated processor 40 to determine that a selected function is to be actuated.

In some versions, once a function that has been activated is deactivated, the function is automatically deselected. Thus, to again activate the function, the clinician must again actuate the associated switch to select the function, and again shift their weight to activate the triggers to actuate the function.

In additional versions, deactivating the selected function does not necessarily deselect that function. And thus the clinician can again actuate the selected function by returning both shoes to the ground and shifting weight forward so that both triggers again measure above the threshold. In such a version, the clinician will take one or more affirmative steps to deselect the selected function, as will be discussed below.

Once the clinician has completed the use of a particular function or wishes to move to a different function of the medical device equipment 60, a deselection step 78 may be required to deselect the function. This can be accomplished in multiple ways. For example, the clinician can actuate the switch corresponding to the selected function so that no function is selected. Also, if the clinician wishes to move to another function, he or she can simply actuates the switch corresponding to another, nonselected function. The selected function will then be deselected and the function corresponding to the actuated switch will be selected. For example, if the first function is selected and the clinician actuates switch S2, the first function will be deselected and the second function will be selected. Also, if the clinician were to, for example, simply walk away from the operating table or medical equipment 60, the movement sensor 50 will signal an indication of such movement to the processor 40. Upon recognition of such movement the processor 40 will deselect whatever function may have been selected. The processor 40 preferably can also be configured to terminate operation of the shoes 20, 22 upon receiving such signals from the movement sensor 50.

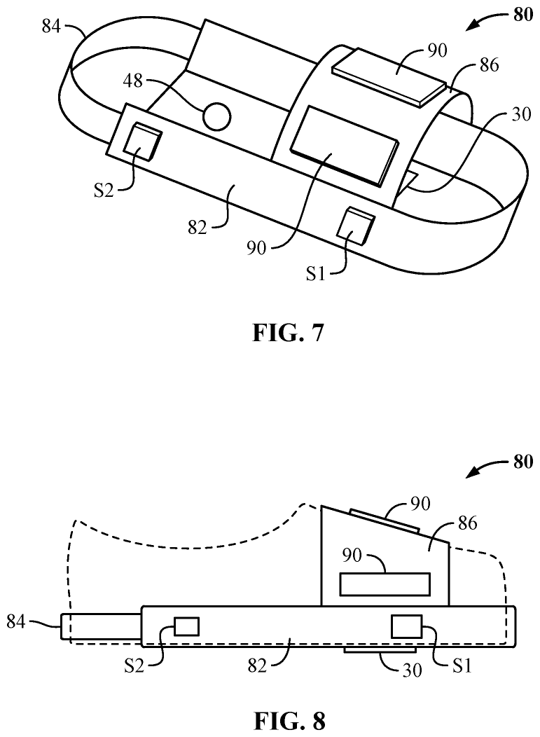

The embodiments discussed above have presented the medical device control system 18 in connection with a pair of shoes 20, 22. It is to understand that that, in additional embodiments, the same or similar structure and functionality can be incorporated into attachments that can be attached to the clinician's own shoes. For example, with reference next to FIGS. 7 and 8, an embodiment of a left shoe attachment 80 comprises a rim 82 configured to generally follow the outline of a typical shoe. A heel portion 84 can be adjustable and/or elastic so that a wearer's shoe of various sizes can be slid within the rim 82 and secured in place by the heel portion 84. An upper portion 86 can above the rim 82 from one side to the other, and preferably is configured to fit generally above the upper of a wearer's own shoe 20a. A bottom 78 can extend between bottom edges of the rim 82. As shown, switches, such as first and second switches S1, S2 can be supported by the rim 82. A trigger sensor 30 and feedback structure 48 can be supported by the bottom 78, and other componentry 90, which can include the processor 40, battery 42 and other desired components, can be supported by the upper portion 86. The illustrated attachment 80 is configured to fit over shoe 20a of the clinician and provide the same functionality as if the components were incorporated into shoes 20, 22. It is anticipated that versions of shoe attachments can take on different structures and configurations.

Additionally, it is anticipated that variations in specific structure can be made while employing the principles discussed herein. For example, another version can employ additional pressure sensors in additional locations, such as aligned with the heel of a shoe, so as to obtain additional sensor data concerning weight distribution of the wearer. Further, threshold calculations can include consideration of other sensors, such as heel sensors.

Further, rather than employ a pressure sensor, some versions may assign function selection functionality to certain switches, while triggering functionality is assigned to another of the switches. For example, switches S1, S2, and S4 can each be tied to selecting a corresponding function when actuated, but switch S3 may be configured to actuate whichever function has been selected. Further, in some versions, actuating the switch S3 once will trigger activation of the selected function of the medical equipment, and the function will remain in an activated state until switch S3 is again activated. In another version, the function will remain in an activated state only so long as the clinician holds the switch S3 in an actuated state.

Still further, the illustrated embodiments have contemplated four switches configured to control up to four functions of the medical equipment. It is to be understood that additional versions can employ more or less switches, and correspondingly be configured to control more of less functions. For example, one additional embodiment may employ only one shoe or shoe attachment and can be configured to control only one or two functions. Further, rather than requiring triggers of both shoes to measure above a threshold, actuation of a selected function can be triggered if only one of the shoes measures pressures within a given range.

In yet another version, the trigger pressure sensors can provide more input than just turning a function on and off. For example, when the trigger measurement exceeds a threshold, the function may be turned on, but if the trigger measurement exceeds a second, higher threshold, the function may increase in its intensity. Thus, the system 18 can facilitate not only on/off control, but also variations in operation such as intensity, speed, power or the like.

In a still further embodiment, instead of employing a two-tier actuation input requirement, a function can be immediately activated and operated upon actuation of a corresponding switch of the shoe.

In versions discussed above, a single function has been correlated to each switch S1-S4. In another version, each switch can have multiple functions correlated therewith, and selection of one of the functions depends on an actuation function of the corresponding switch. For example, tapping the switch one time may direct selection of a first function while tapping the switch twice in succession can direct selection of a second function. The feedback structure can be configured to verify to the clinician as to which function has been selected. For example, selection of the first function is signaled by providing a first haptic feedback pattern, while selection of the second function is signaled by providing a second haptic feedback pattern.

The operation of the shoe was described in relation to a threshold pressure as a percentage of the person's weight. However, it is also contemplated that the function can be actuated when the pressure sensed by the sensor increases by a threshold percentage after the switch (S1, S2, S3 or S4) being activated has been activated. By way of example and not limitation, when the clinician stands on the left foot and moves the right shoe toward the left shoe near switch S1, the trigger sensor in the left shoe senses pressure. When the right shoe is placed back on the ground, the trigger sensor in the left shoes senses a reduction in pressure since the right foot is now taking some of the clinician's weight. To actuate function 1, the clinician can lean forward to increase pressure applied to the trigger sensor. The increased pressure if it exceeds a threshold percentage change (e.g., 20%) can actuate the function 1 of the device. If the increased pressure does not exceed the threshold percentage change (e.g., 20%, then the function 1 of the device is not activated. This alternate description of the operation of the shoe may be used in combination with any and all other aspects described above.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited specific combination described herein. Thus, the scope of the claims is not to be limited by the illustrated embodiments.

What is claimed is:

1. A medical system, comprising:
a medical equipment having a function, the medical equipment configured so that the function can be changed from a first operational state to a second operational state;
first and second shoes having an electronic interface in communication with the medical equipment; and
a switching mechanism attached to one or more of the first and second shoes, the switching mechanism comprising:
a first switch attached to the first shoe;
a processor in communication with the first switch,
a transceiver in communication with the processor and the medical equipment;
the first switch actuatable when the second shoe is put in contact or close proximity with the first shoe;
a trigger sensor disposed on a sole of the first shoe and operative to sense a downward pressure applied by a first foot of a user, the trigger sensor configured to communicate downward pressure data to the processor;
the processor configured to actuate a first function of the medical equipment upon receiving downward pressure data reflecting an increase in pressure after actuation of the first switch; and
the transceiver configured to wirelessly transmit a first control instruction to the system interface for actuating the first function of the medical equipment;
wherein the medical equipment is configured to transition the first function from the first operational state to the second operational state in response to receipt of the first control instruction.

2. The medical system of claim 1, wherein the switching mechanism is embedded into the first shoe, second shoe or both first and second shoes.

3. The medical system of claim 1, wherein the switching mechanism additionally comprises a second switch in the first shoe and configured to be actuated when the second shoe is put in contact or close proximity with the first shoe.

4. The medical system of claim 3, wherein the processor is configured to select a second function upon receiving a second function actuation signal, and the processor is configured to generate a second control instruction when data from the trigger sensor satisfies a trigger criteria and the second function is selected.

5. The medical system of claim 4 further comprising a second switching mechanism incorporated into the second shoe, the second switching mechanism comprising a third sensor, a second processor, and a second transceiver.

6. The medical system of claim 1, wherein the switching mechanism additionally comprises a speaker or vibrator configured to provide feedback to the user of the first and second shoes when the first function of the medical equipment has been selected or actuated.

7. A method for hands-free control of a medical equipment, comprising:
receiving a primary input from a clinician, the primary input comprising the clinician actuating a first input structure carried on a first shoe of the clinician to select a first function of the medical equipment;
generating a feedback to notify the clinician that the first function has been selected;
receiving a secondary input from the clinician, the secondary input comprising the clinician actuating a trigger input structure carried on the first shoe of the clinician;
placing the selected first function of the medical equipment in an operational state upon receiving the secondary input; and
maintaining the selected first function of the medical equipment in the operational state for as long as the trigger input structure is actuated.

8. The method of claim 7, wherein the trigger input structure comprises a pressure sensor for sensing a downward pressure applied by a weight of the clinician.

9. The method of claim 8, further comprising actuating the trigger input structure when the clinician shifts his or her weight forwardly onto balls of feet of the clinician.

10. The method of claim 9, further comprising determining that the trigger input structure is actuated when a pressure data from the pressure sensor satisfies an actuation criteria.

11. The method of claim 10, further comprising receiving a second primary input from the clinician, the second primary input comprising the clinician actuating a second input structure carried on a second shoe of the clinician, and in response to the second primary input de-selecting the first function and selecting a second function of the medical equipment.

12. The method of claim 11, wherein receiving the secondary input comprises the clinician actuating the trigger input structure carried on the first shoe and actuating a second trigger input structure carried on the second shoe of the clinician, the second trigger input structure comprising a second pressure sensor, a second processor carried on the second shoe of the clinician determining that the second trigger input structure is actuated when a second pressure data from the second pressure sensor satisfies the actuation criteria.

13. The method of claim 12, additionally comprising the second processor electronically communicating that the second pressure sensor satisfies the actuation criteria to the processor of the first shoe.

14. The method of claim 12, comprising placing the second function of the medical equipment in an operational state when the pressure sensor of the first shoe satisfies the actuation criteria and the second pressure sensor also satisfies the actuation criteria.

15. The method of claim 7, wherein generating the feedback comprises activating a haptic feedback that can be felt by a foot of the clinician wearing the first shoe.
